(19) United States
(12) Patent Application Publication
(10) Pub. No.: US 2022/0228137 A1
(43) Pub. Date: Jul. 21, 2022

(54) METHOD, COMPOSITION AND KIT FOR SIZE SELECTIVE ENRICHMENT OF NUCLEIC ACIDS

(71) Applicant: PHASE SCIENTIFIC INTERNATIONAL, LTD., Hong Kong (CN)

(72) Inventors: Yin To CHIU, Hong Kong (CN); Daniel Robert MARSHAK, Weston, MA (US); Harsha Madan KITTUR, Huntingtown, MD (US); Masae KOBAYASHI, Buena Park, CA (US)

(21) Appl. No.: 17/753,178

(22) PCT Filed: Aug. 26, 2020
(86) PCT No.: PCT/CN2020/111449

§ 371 Date: Feb. 22, 2022

Related U.S. Application Data
(60) Provisional application No. 62/892,041, filed on Aug. 27, 2019.

(51) Int. Cl. C12N 15/10; C12Q 1/6806

(52) U.S. Cl. CPC C12N 15/1006; C12Q 1/6806

(57) ABSTRACT

Provided is a method for isolating and concentrating nucleic acids of selected target sizes (e.g., in increments less than 1000 base pairs) from a biological fluid mixture comprising combining the biological fluid mixture and a first aqueous two-phase system (ATPS) formed from a first phase forming polymer or surfactant component dissolved in a first phase solution, and a second phase solution, such that target nucleic acid fragments below a desired target size partition to said second phase solution and contaminants partition to the first phase solution, extracting and mixing the second phase solution with a second ATPS formed from a second phase forming polymer or surfactant component dissolved in a third phase solution and a fourth phase solution, such that the target nucleic acid fragments partition to and concentrate in the third phase solution, and recovering the concentrated target nucleic acid fragments from the third phase solution. A composition and kit for isolating and concentrating nucleic acids of selected target sizes as described above are also provided.

### FIELD OF THE INVENTION

This invention relates to the isolation, concentration and/or purification of nucleic acid fragments in sequential aqueous phase systems. In particular, the present invention provides sample preparation methods, compositions and kit components for the isolation, concentration and/or purification of nucleic acid fragments from biological materials.

### BACKGROUND

Methods of isolation and purification of nucleic acids (such as DNA and RNA) from complex matrices such as blood, tissue, urine and forensic samples, and bacterial and mammalian cell culture media, are important in genetic research, nucleic acid probe diagnostics, forensic DNA testing and other areas that require amplification, processing or analysis of nucleic acids. Purified nucleic acids must be of high quality and reach sufficient quantity, such that they can be used in various downstream applications including detection, sequencing, clinical diagnosis and the like. Obtaining purified nucleic acids is a complicated task due to the presence of large amounts of contaminating cellular materials, (e.g. proteins and carbohydrates) present in the complex environments in which the nucleic acids are identified, including urine, blood, plasma, serum, saliva and other biological fluids. Current methods for extraction and purification of nucleic acids from biological samples are usually time consuming, tedious, costly, involve the use of hazardous organic solvents, and often are suitable for capturing only nucleic acids above certain sizes, for example, 1000 bp.

For target analytes that are present at very low concentrations in biological fluids, such as urine and blood, there is a need to obtain large volumes of biological fluids in order to obtain sufficient quantity of the target analytes for subsequent detection by molecular techniques. It has been a significant challenge to detect the existence of an analyte which has an extremely low concentration. The analyte can be a biomarker of a disease such as a cell free DNA (cfDNA), circulating tumor DNA (ctDNA) or a protein which exists in a sample such as saliva, blood, urine and other bodily fluids of a patient. Many of the existing diagnostic or detection methods may falsely report that the analyte does not exist if the analyte concentration is too low. For instance, the gold standard of diagnostics such as Polymerase Chain Reaction (PCR) and Enzyme-Linked Immune Sorbent Assay (ELISA) may produce a false negative result if the target analyte has extremely low quantity beyond the detection limit of the assay.

Where biomarkers are present in low quantities, accurate detection hinges upon isolation methods that can concentrate the nucleic acids from background. Depending on the isolation method, this challenge can be complicated with variance in nucleic acid fragment size and influences by test inhibitors. There are a variety of methods that have been used for the purification of nucleic acids. These include precipitation, ultrafiltration (Hirasaki et al., J. Membr. Sci., 106: 123-129 (1995)) and also adsorption using anion-exchange columns. Other methods tested including commercially available methods are reported to have lower DNA extraction yields and to fail to extract small DNA fragments of less than 200 bp in length (Fong et al, Clinical Chemistry 55(3), 587-589, 2009). Ribeiro et al. (Biotechnol. Bioeng. May 20, 2002; 78(4):376-84) describe an aqueous 2-phase system (ATPS) with PEG as the polymer component and dipotassium hydrogen phosphate (K₂HPO4) as the salt component. The isolation of the plasmid pCF1-CFTR from E. coli DH5a is reported. For this, the cells were first broken down by means of alkaline lysis (lysis buffer containing NaOH and SDS) and the lysate was then neutralized with 3 M sodium acetate. The cell debris, proteins and genomic DNA (gDNA) were subsequently removed by centrifugation of the batch. The clarified lysate was employed in the abovementioned aqueous 2-phase system. However, this process has the disadvantages that it is very expensive in terms of apparatus and time. The aforementioned articles are hereby incorporated by reference in their entirety. Commercial bench-top solid-phase kits, such as those provided by Qiagen® and Invitrogen®, allow relatively fast extraction and purification of nucleic acids, but require bench-top laboratory equipment, meaning that the extraction and purification is largely limited to laboratories with fundamental setup and power sources.

A simplified method and device to achieve higher concentration and higher number of molecules of nucleic acids of small target fragment sizes in a precise, higher signal to noise for downstream applications, inexpensive manner compatible with various industrial, clinical and research uses, such as point-of-care testing, would therefore be highly desired.

### SUMMARY OF THE INVENTION

To overcome the aforementioned limitations of existing technologies, disclosed are novel methods, compositions and kits for size selective enrichment, isolation and analyses of analytes in an inexpensive, fast, and entirely liquid process employing sequential aqueous two-phase systems (ATPS) without the need of complex equipment. The methods and compositions can achieve the following multiple tasks, including cell lysis, removing non-targeted biomolecules, and/or concentrating targeted analytes. In some embodiments, the analyte is a nucleic acid fragment below a selected size, e.g., a nucleic acid comprising fewer than 1000 basepairs (e.g., fewer than 10,000 bp, 1000 bp, 900 bp, 800 bp, 700 bp, 600 bp, 500 bp, 450 bp, 400 bp, 350 bp, 300 bp, 250 bp, 200 bp, 150 bp, 100 bp, or 50 bp.) In some embodiments, the analyte is single-stranded nucleic acid, while in other embodiments the analyte is a double-stranded nucleic acid. Target size cut-offs may be selected through appropriate selection and specific ordering of ATPS phase forming and fractionating components, and centrifugation, mixing and incubation steps. In some embodiments, nucleic acid fragments can be isolated and concentrated with 50 bp target size cut-off precision below 1000 bp, and more preferably with 25 bp precision, in the ranges most beneficial for analyses.

The invention provides an improved method for purifying nucleic acids from biological samples and then removing components of the ATPS from the nucleic acids that would otherwise interfere with downstream applications such as disease detection, amplification and genotyping. Exemplary biological sample may comprise blood, plasma, saliva, urine, cells, exosomes, proteins, cfDNA, RNA and circulating tumor cells. The inventors unexpectedly discovered that the disclosed stable, repeatable processes accomplish nucleic acid purification with little to no loss of nucleic acid (i.e., very high recovery), and that that by extracting one phase of a unique ATPS, mixing it with phase forming components that have different chemical properties than the phase forming components of the first ATPS, a second ATPS may be formed where the target nucleic acids will partition to a phase in the second ATPS opposite of the phase they partitioned to in the first ATPS.

This provided in some embodiments is a method is disclosed for isolating and concentrating nucleic acids of desired target sizes from a fluid biological mixture including nucleic acids and contaminants. The biological mixture may be combined with a first ATPS formed from a first phase forming polymer or surfactant component dissolved in a first phase solution, and a second phase solution, such that target nucleic acid fragments below a target size are isolated by partitioning to said second phase solution while contaminants partition to the first phase solution. The second phase solution may then be extracted and mixed with a second ATPS formed from a second phase forming polymer or surfactant component dissolved in a third phase solution and a fourth phase solution, such that the target nucleic acid fragments are concentrated by partitioning to the third phase solution. The concentrated target nucleic acid fragments may then be recovered from the third phase solution in any number of ways.

In some embodiments, the first and second phase forming polymer or surfactant components may comprise one or more polymer, one or more surfactant, and combinations thereof. Possible polymers that may be employed include, but are not limited to, polyalkylene glycols (PEGs), such as hydrophobically modified polyalkylene glycols, poly(oxyalkylene)polymers, poly(oxyalkylene)copolymers, such as hydrophobically modified poly(oxyalkylene)copolymers, polyvinyl pyrrolidone, polyvinyl alcohol, polyvinyl caprolactam, polyvinyl methylether, alkoxylated surfactants, alkoxylated starches, alkoxylated cellulose, alkyl hydroxyalkyl cellulose, silicone-modified polyethers, and poly N-isopropylacrylamide and copolymers thereof. In another embodiment, the first phase forming polymer comprises polyethylene glycol, polypropylene glycol, or dextran. Possible surfactants that may be employed include but are not limited to Triton-X, Triton-114, Igepal CA-630 and Nonidet P-40, anionic surfactants, such as carboxylates, sulphonates, petroleum sulphonates, alkylbenzenesulphonates, naphthalenesulphonates, olefin sulphonates, alkyl sulphates, sulphates, sulphated natural oils & fats, sulphated esters, sulphated alkanolamides, alkylphenols, ethoxylated and sulphated, nonionic surfactants, such as ethoxylated aliphatic alcohol, polyoxyethylene surfactants, carboxylic esters, polyethylene glycol esters, anhydrosorbitol ester, glycol esters of fatty acids, carboxylic amides, monoalkanolamine condensates, polyoxyethylene fatty acid amides, cationic surfactants, such as quaternary ammonium salts, amines with amide linkages, polyoxyethylene alkyl & alicyclic amines, n,n,n′,n′ tetrakis substituted ethylenediamines, 2-alkyl 1-hydroxethyl 2-imidazolines, and amphoteric surfactants, such as n-coco 3-aminopropionic acid/sodium salt, n-tallow 3-iminodipropionate, disodium salt, n-carboxymethyl n dimethyl n-9 octadecenyl ammonium hydroxide, n-cocoamidethyl n hydroxyethylglycine, and sodium salt.

In some embodiments, the first polymer or surfactant component has a higher molecular weight than the second polymer or surfactant component.

In some embodiments the molar concentration of the first polymer or surfactant component in the first phase solution of the NA-isolating ATPS is higher than the molar concentration of at least one of the second polymer or surfactant component in the third phase solution, the second polymer or surfactant component in the fourth phase solution, and the second polymer or surfactant component in the NA-concentrating ATPS.

In some embodiments, the mass concentration of the first polymer or surfactant component in the first phase solution of the NA-isolating ATPS is higher than the mass concentration of at least one of the second polymer or surfactant component in the third phase solution, the second polymer or surfactant component in the fourth phase solution, and the second polymer or surfactant component in the NA-concentrating ATPS. In one embodiment, the first polymer or surfactant component concentration of the first phase solution is in the range of about 0.01% to about 90% by weight of the total weight of the aqueous solution (w/w). In various embodiments, the first phase solution is selected from a polymer or surfactant solution that is about 0.01% w/w, about 0.05% w/w, about 0.1% w/w, about 0.15% w/w, about 0.2% w/w, about 0.25% w/w, about 0.3% w/w, about 0.35% w/w, about 0.4% w/w, about 0.45% w/w, about 0.5% w/w, about 0.55% w/w, about 0.6% w/w, about 0.65% w/w, about 0.7% w/w, about 0.75% w/w, about 0.8% w/w, about 0.85% w/w, about 0.9% w/w, about 0.95% w/w, or about 1% w/w. In some embodiments, the first phase solution is selected from polymer or surfactant solution that is about 1% w/w, about 2% w/w, about 3% w/w, about 4% w/w, about 5% w/w, about 6% w/w, about 7% w/w, about 8% w/w, about 9% w/w, about 10% w/w, about 11% w/w, about 12% w/w, about 13% w/w, about 14% w/w, about 15% w/w, about 16% w/w, about 17% w/w, about 18% w/w, about 19% w/w, about 20% w/w, about 21% w/w, about 22% w/w, about 23% w/w, about 24% w/w, about 25% w/w, about 26% w/w, about 27% w/w, about 28% w/w, about 29% w/w, about 30% w/w, about 31% w/w, about 32% w/w, about 33% w/w, about 34% w/w, about 35% w/w, about 36% w/w, about 37% w/w, about 38% w/w, about 39% w/w, about 40% w/w, about 41% w/w, about 42% w/w, about 43% w/w, about 44% w/w, about 45% w/w, about 46% w/w, about 47% w/w, about 48% w/w, about 49% w/w, and about 50% w/w.

In some embodiments, the second phase solution in the isolating ATPS includes at least one phase forming dissolved surfactant or polymer component as noted above, and/or a dissolved salt such as dipotassium phosphate, monopotassium phosphate, and combinations thereof. In some embodiments, the salt includes, but is not limited to, kosmotropic salts, chaotropic salts, inorganic salts containing cations such as straight or branched trimethyl ammonium, triethyl ammonium, tripropyl ammonium, tributyl ammonium, tetramethyl ammonium, tetraethyl ammonium, tetrapropyl ammonium and tetrabutyl ammonium, and anions such as phosphates, sulphate, nitrate, chloride and hydrogen carbonate. In another embodiment, the salt may comprise NaCl, Na₃PO₄, K₃PO₄, Na₂SO₄, potassium citrate, (NH₄)₂SO₄, sodium citrate, sodium acetate and combinations thereof. Other salts, e.g. ammonium acetate, may also be used. In another embodiment, the salt may be selected from magnesium salt, a lithium salt, a sodium salt, a potassium salt, a cesium salt, a zinc salt and an aluminum salt. In some embodiments, the salt may be selected from a bromide salt, an iodide salt, a fluoride salt, a carbonate salt, a sulfate salt, a citrate salt, a carboxylate salt, a borate salt, and a phosphate salt. In some embodiments, the salt comprises potassium phosphate. In some embodiments, the salt comprises ammonium sulfate. In one embodiment, the total salt concentration is in the range of about 0.01% to about 90%. A skilled person in the art will understand that the amount of salt needed to form an ATPS will be influenced by molecular weight, concentration and physical status of the polymer or surfactant. In various embodiments, the salt solution is about 0.001% to 90% w/w. In various embodiments, the salt solution is about 0.01% w/w, about 0.05% w/w, about 0.1% w/w, about 0.15% w/w, about 0.2% w/w, about 0.25% w/w, about 0.3% w/w, about 0.35% w/w, about 0.4% w/w, about 0.45% w/w, about 0.5% w/w, about 0.55% w/w, about 0.6% w/w, about 0.65% w/w, about 0.7% w/w, about 0.75% w/w, about 0.8% w/w, about 0.85% w/w, about 0.9% w/w, about 0.95% w/w, or about 1% w/w. In some embodiments, the salt solution is about 1% w/w, about 2% w/w, about 3% w/w, about 4% w/w, about 5% w/w, about 6% w/w, about 7% w/w, about 8% w/w, about 9% w/w, about 10% w/w, about 11% w/w, about 12% w/w, about 13% w/w, about 14% w/w, about 15% w/w, about 16% w/w, about 17% w/w, about 18% w/w, about 19% w/w, about 20% w/w, about 21% w/w, about 22% w/w, about 23% w/w, about 24% w/w, about 25% w/w, about 26% w/w, about 27% w/w, about 28% w/w, about 29% w/w, about 30% w/w, about 31% w/w, about 32% w/w, about 33% w/w, about 34% w/w, about 35% w/w, about 36% w/w, about 37% w/w, about 38% w/w, about 39% w/w, about 40% w/w, about 41% w/w, about 42% w/w, about 43% w/w, about 44% w/w, about 45% w/w, about 46% w/w, about 47% w/w, about 48% w/w, about 49% w/w, and about 50% w/w. In one embodiment, the concentration of salt is about 2% to 40% w/w. In one embodiment, the concentration of salt is about 3% to 30% w/w. In one embodiment, the concentration of salt is about 5% to 20% w/w.

In some embodiments, the fourth phase solution in the concentrating ATPS includes at least one phase forming dissolved salt, surfactant or polymer component as described above, and combinations thereof.

In some embodiments, the second phase solution of the isolating ATPS exerts weaker excluded volume interactions upon the target nucleic acid fragments than the fourth phase solution of the concentrating ATPS. In some embodiments, the first phase solution of the isolating ATPS exerts stronger excluded volume interactions upon the target nucleic acid fragments than the third phase solution of the concentrating ATPS.

In some embodiments, the second phase solution of the isolating ATPS exerts hydrophilic and hydrophobic interactions more favorable to partitioning of the target nucleic acid fragments into said second phase than the fourth phase solution of the concentrating ATPS. More specifically, there is a favorable change in free energy when a molecule of target DNA moves from the first phase solution into the second phase solution than when a molecule of DNA moves from the third phase solution to the fourth phase solution. In some embodiments, the first phase solution of the isolating ATPS exerts hydrophilic and hydrophobic interactions less favorable to partitioning of the target nucleic acid fragments into said first phase solution than the third phase solution of the concentrating ATPS.

In some embodiments, the second phase solution of the isolating ATPS exerts electrostatic interactions more favorable to partitioning of the target nucleic acid fragments into said second phase than the fourth phase solution of the concentrating ATPS. More specifically, there is a favorable change in free energy when a molecule of target DNA moves from the first phase solution into the second phase solution than when a molecule of DNA moves from the third phase solution to the fourth phase solution. In some embodiments, the first phase solution of the isolating ATPS exerts electrostatic interactions less favorable to partitioning upon the target nucleic acid fragments into said first phase solution than the fourth phase solution of the concentrating ATPS.

In one specific embodiment, recovery of the concentrated target nucleic acid fragments in the second ATPS may comprise separating the third phase solution from the fourth phase solution, and mixing the third phase solution with at least one size fractionation component selected from a polymer, a surfactant, a salt, and combinations thereof in order to form a supernatant comprised of the concentrated target nucleic acids and a precipitated pellet of nucleic acids above the target cut-off size. Then the supernatant may be separate from the precipitated pellet of nucleic acids above the target cut-off size and the target nucleic acid fragments below the selected cut-off size may be precipitated from the supernatant.

In some embodiments, the size fractionating component may comprise a polymer or surfactant component having a higher molecular weight than the first polymer or surfactant component of the isolating ATPS, where the first polymer or surfactant component has a higher molecular weight than the second polymer or surfactant component of the concentrating ATPS.

In some embodiments, the molar concentration of the at least one size fractionating component in the supernatant may be less than the molar concentration of the first polymer or surfactant component in the first phase solution, and the first polymer or surfactant component in the first phase solution has a higher molar concentration than the molar concentration of the second polymer or surfactant component in the third phase solution.

In some embodiments, the mass concentration of the at least one size fractionating component in the supernatant is less than the mass concentration of the first polymer or surfactant component in the first phase solution, and the first polymer or surfactant component in the first phase solution has a mass concentration higher than the mass concentration of the second polymer or surfactant component in the third phase solution.

In some embodiments, the at least one size fractionating component may comprise one or more salt, polymer and/or surfactant such as described above, or combinations thereof.

In another major embodiment, a composition is disclosed for isolating and concentrating nucleic acids of selected small target sizes from a fluid (e.g., biological) mixture including nucleic acids and contaminants. The composition may include components for forming a NA-isolating ATPS from a first phase forming polymer or surfactant component dissolved in a first phase solution, and a second phase solution, such that when mixed with the fluid mixture target nucleic acid fragments below a target size partition to said second phase solution and contaminants partition to the first phase solution, and components for forming a NA-concentrating ATPS from a second phase forming polymer or surfactant component dissolved in a third phase solution and a fourth phase solution, such that when mixed with the second phase solution the target nucleic acid fragments partition to and concentrate in the third phase solution. The composition may also include materials for concentrating the target nucleic acid fragments from the third phase solution.

In yet another embodiment, a kit is disclosed for isolating and concentrating nucleic acids of target sizes from a fluid mixture including nucleic acids and contaminants. The kit may include the composition components described in the composition embodiment, but additionally syringe or pipette accessible containers for storage, packing, and/or reactions and optionally equipment for manipulating the aqueous solutions. Such containers and equipment may include columns, test tubes capillary tubes, plastic test tubes, falcon tubes, culture tubes, well plates, pipettes and/or cuvettes

These and other features and characteristics, as well as the methods of operation and functions of the related components and economies of manufacture, will become more apparent upon consideration of the following detailed description and the appended claims with reference to the accompanying figures, all of which form a part of this specification, wherein like reference numerals designate corresponding parts in the various figures. It is to be expressly understood, however, that the drawings are for the purpose of illustration and description only and are not intended as a definition of the limits of the claims. As used in the specification and in the claims, the singular form of “a”, “an”, and “the” include plural referents unless the context clearly dictates otherwise

### BRIEF DESCRIPTION OF THE DRAWING

FIG. 1 illustrates an embodiment of a workflow for isolating and concentrating nucleic acids of small fragment sizes from a fluid (e.g., biological) mixture.

FIG. 2 shows a gel electrophoresis image of nucleic acids partitioned by one embodiment of the invention.

FIGS. 3A and 3B show the percentage of nucleic acid recovered in embodiments of the isolation and concentration steps according to the invention.

FIG. 4 shows gel electrophoresis images of nucleic acid size fractionation into pellets and supernatants in accordance with several embodiments of the invention.

FIG. 5 shows gel electrophoresis images of nucleic acid fractionation into pellets and supernatants by selected fragment sizes in accordance with several embodiments of the invention.

FIG. 6 shows comparative DNA recovery between an embodiment of the invention and a Qiagen kit.

FIG. 7 shows gel electrophoresis image of the top and bottom phases in accordance with an example embodiment of the invention.

FIG. 8 shows the DNA recovery and the protein recovery of three samples in accordance with an example embodiment of the invention.

FIG. 9 shows the gel electrophoresis image of the liquids removed, final extractions and T-rich phases in accordance with an example embodiment of the invention.

FIG. 10 shows the gel electrophoresis image of the pellets and the supernatants in accordance with several embodiments of the invention.

### DETAILED DESCRIPTION

Unless indicated otherwise, the terms used herein, including technical and scientific terms, have the same meaning as usually understood by those skilled in the art to which the present invention pertains and detailed descriptions of well-known functions and constitutions that may obscure the gist of the present invention are omitted.

‘Aqueous,’ as used herein, refers to the characteristic properties of a solvent/solute system wherein the solvating substance has a predominantly hydrophilic character. Examples of aqueous solvent/solute systems include those where water, or compositions containing water, are the predominant solvent. The polymer and/or surfactant components whose use is described in the embodiments are “aqueous” in the sense that they form aqueous phases when combined with a solvent such as water. Further, as understood by the skilled person, in the present context the term liquid “mixture” refers merely to a combination of the herein-defined components.

As used herein, an aqueous two-phase system (ATPS) means a liquid-liquid separation system that can accomplish isolation or concentration of an analyte by partitioning, where two phases, sections, areas, components, or the like, interact differently with at least one analyte to which they are exposed and optionally dissolved. An ATPS is formed when two immiscible phase forming components, such as a salt and polymer, or two incompatible polymers (e.g., PEG and dextran) with certain concentration are mixed in an aqueous solution. ATPS methods are relatively inexpensive and scalable because they employ two-phase partitioning to separate analytes (e.g., nucleic acids) from contaminants.

The term ‘isolated’ as used herein refers to nucleic acid removed from its original environment and thus is altered from its original environment. An isolated nucleic acid generally is provided with fewer non-nucleic acid components (e.g., protein, lipid) than the amount of components present in a source sample. A composition comprising isolated sample nucleic acid can be substantially isolated (e.g., about 90%, 91%, 92%, 93%, 94%, 95%, 96%, 97%, 98%, 99% or greater than 99% free of non-nucleic acid components).

As used herein, ‘concentrated’ means that the mass ratio of analyte in question to the solution in which the analyte is suspended is higher than the mass ratio of said analyte in its pre-concentration solution. It can, for example, be slightly higher, or more preferably at least twice, ten times or one hundred times as high.

As used herein, ‘polymer’ includes, but is not limited to, homopolymer, copolymer, terpolymer, random copolymer, and block copolymer. Block copolymers include, but are not limited to, block, graft, dendrimer, and star polymers. As used herein, copolymer refers to a polymer derived from two monomeric species; similarly, a terpolymer refers to a polymer derived from three monomeric species. The polymer also includes various morphologies, including, but not limited to, linear polymer, branched polymer, random polymer, crosslinked polymer, and dendrimer systems. As an example, polyacrylamide polymer refers to any polymer including polyacrylamide, e.g., a homopolymer, copolymer, terpolymer, random copolymer, block copolymer or terpolymer of polyacrylamide. Polyacrylamide can be a linear polymer, branched polymer, random polymer, crosslinked polymer, or a dendrimer of polyacrylamide.

Preparation of Sample

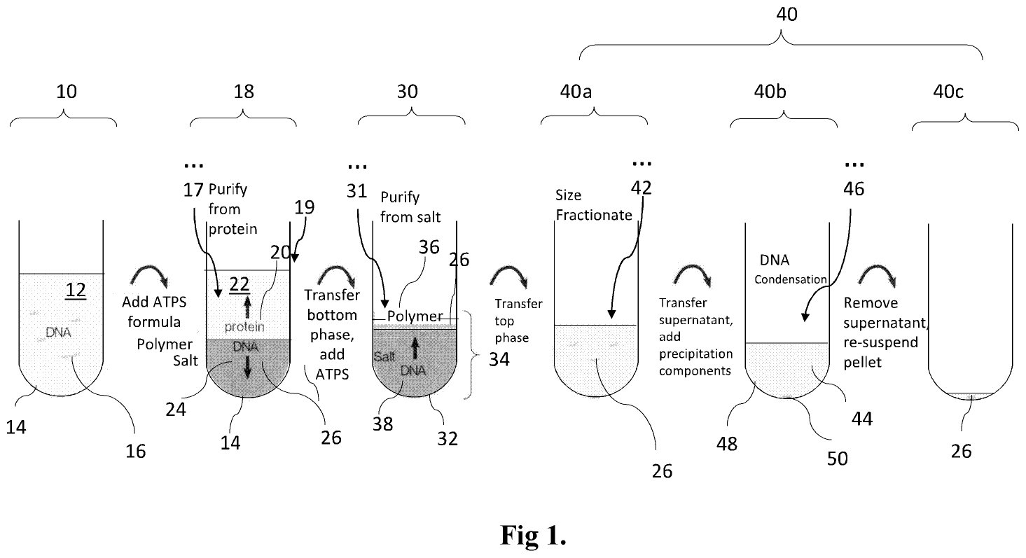

With reference to FIG. 1, an example method embodiment may include an optional first step 10 of preparing a fluid mixture 12 by mixing in a suitable vessel 14 a biological sample and a lysis buffer and incubating for a sufficient time (e.g., preferably within a range of 1 to 60 minutes) at a proper temperature (e.g., in a range from 15° to 40° C.) to release nucleic acids 16 from cells, exosomes, proteins and/or other materials in the biological sample. Preferably the lysis buffer may have a pH in the range of from 4 to 11, preferably of from 7 to 10 and most preferably of from 8 to 9. The concentration of substances in the lysis buffer depends on the amount of biologic material to be lysed and the manner of the provision of said biologic material. In principle, any separating methods known to the skilled worker may be suitable for releasing nucleic acids from the biological sample. Lysis methods which may be contemplated are in particular lysis by the action of heat, lysis by the action of mechanical force, lysis by enzymes such as, for example, protein kinase K, or lysis by contacting the cells to a lysis buffer containing a detergent or a chaotropic compound, or by means of hypotonic solutions. Where appropriate, the abovementioned measures may also be combined, for example by mechanically disrupting the cells in a lysis buffer containing a detergent or a chaotropic compound or, for example, by employing a lysis buffer containing protein kinase K together with a chaotropic compound.

Isolation of Analyte

Isolation components 17 may be added into the mixture 12, in an isolation step 18, forming an aqueous two-phase system, ATPS 19, with or without centrifugation, in order to separate target nucleic acid fragments 26 from contaminants 20. The isolation components 17 may include a first phase forming polymer or surfactant component dissolved in a first phase solution 22, and a second phase solution 24, such that the target nucleic acid fragments 26 below a selected target cut-off size partition into the second phase solution 24 (e.g., a salt-rich lower phase), while proteins and other contaminants 20 partition to the first phase solution 22 (e.g., a polymer-rich upper phase.)

A number of types of ATPS 19 may be utilized, including polymer-salt systems, polymer-polymer systems, polymer-surfactant systems, and micellar or reverse micellar systems. Phase separation partitioning may be affected by factors such as appropriate selection and specific ordering of the isolation components 17, pH, molecular weight, relative concentrations, as well as centrifugation, mixing and incubation steps.

The isolation components 17 may include polymer or surfactant components that assist in forming first phase solution 22 and second phase solution 24. Suitable polymers may include polyethylene glycol, polypropylene glycol, dextran, polyalkylene glycols, such as hydrophobically modified polyalkylene glycols, poly(oxyalkylene)polymers, poly(oxyalkylene)copolymers, such as hydrophobically modified poly(oxyalkylene)copolymers, polyvinyl pyrrolidone, polyvinyl alcohol, polyvinyl caprolactam, polyvinyl methylether, alkoxylated surfactants, alkoxylated starches, alkoxylated cellulose, alkyl hydroxyalkyl cellulose, silicone-modified polyethers, and poly N-isopropylacrylamide and copolymers thereof. Suitable surfactants include, but are not limited, to Triton-X, Triton-114, Igepal CA-630 and Nonidet P-40, anionic surfactants such as carboxylates, sulphonates, petroleum sulphonates, alkylbenzenesulphonates, naphthalenesulphonates, olefin sulphonates, alkyl sulphates, sulphates, sulphated natural oils & fats, sulphated esters, sulphated alkanolamides, alkylphenols, ethoxylated & sulphated, nonionic surfactants such as ethoxylated aliphatic alcohol, polyoxyethylene surfactants, carboxylic esters, polyethylene glycol esters, anhydrosorbitol ester, glycol esters of fatty acids, carboxylic amides, monoalkanolamine condensates, polyoxyethylene fatty acid amides, cationic surfactants such as quaternary ammonium salts, amines with amide linkages, polyoxyethylene alkyl & alicyclic amines, n,n,n′,n′ tetrakis substituted ethylenediamines, 2-alkyl 1-hydroxethyl 2-imidazolines, and amphoteric surfactants such as n-coco 3-aminopropionic acid/sodium salt, n-tallow 3-iminodipropionate, disodium salt, n-carboxymethyl n dimethyl n-9 octadecenyl ammonium hydroxide, n-cocoamidethyl n hydroxyethylglycine, and sodium salt.

In a polymer-salt embodiment of ATPS 19, second phase solution 24 may include an isolation component comprising a dissolved salt. Suitable salts include, but are not limited to kosmotropic salts, chaotropic salts, inorganic salts containing cations such as straight or branched trimethyl ammonium, triethyl ammonium, tripropyl ammonium, tributyl ammonium, tetramethyl ammonium, tetraethyl ammonium, tetrapropyl ammonium and tetrabutyl ammonium, anions such as phosphates, sulphate, nitrate, chloride and hydrogen carbonate, NaCl, Na3PO4, K3PO4, Na2SO4, potassium citrate, (NH4)2SO4, sodium citrate, sodium acetate, ammonium acetate, magnesium salt, lithium salt, sodium salt, potassium salt, cesium salt, zinc salt, aluminum salt, bromide salt, iodide salt, fluoride salt, carbonate salt, sulfate salt, citrate salt, carboxylate salt, borate salt, phosphate salt, potassium phosphate, ammonium sulfate, and combinations thereof.

In some embodiments, the first phase solution 24 is formed containing an isolation component 17 comprising a polymer having a mean molecular weight of between 200 and 10000 Da (e.g., 200, 300, 400, 500, 600, 700, 800, 900, 1000, 1100, 1200, 1300, 1400, 1500, 1600, 1700, 1800, 1900, 2000, 2100, 2200, 2300, 2400, 2500, 2600, 2700, 2800, 2900, 3000, 3100, 3200, 3300, 3400, 3500, 3600, 3700, 3800, 3900, 4000, 4100, 4200, 4300, 4400, 4500, 4600, 4700, 4800, 4900, 5000, 5100, 5200, 5300, 5400, 5500, 5600, 5700, 5800, 5900, 6000, 7000, 8000, 9000, 10000, 20000, 30000, 50000, or 100000 Da.)

Concentration of Analyte

It is desirable to decrease the process volume of the working solution containing the nucleic acid fragments 26. In an ATPS, this can be achieved by decreasing the volume of the top-phase. In some embodiments of concentrating step 30, the second phase solution 24 containing target nucleic acid fragments 26 may be extracted from vessel 14 and mixed with concentration components 31 in a second vessel 32, with or without centrifugation, in order to form a second ATPS 34. In other embodiments, the nucleic acid poor first phase solution 22 may be extracted, and vessel 14 may be used in performing concentrating step 30. The concentration components 31 may comprise a second phase forming polymer or surfactant component dissolved in a third phase solution 36 (e.g., a top phase) and a fourth phase solution 38 (e.g., a bottom phase), such that the target nucleic acid fragments 26 partition into and concentrate in the third phase solution 36 (e.g., a polymer-rich upper phase), while salt and other contaminants partition to the fourth phase solution 38 (e.g., a salt-rich bottom phase.)

In some embodiments, nucleic acid fragments 26 may be concentrated into 1/10 the aqueous solution volume compared to the pre-concentration aqueous bottom phase volume extracted from vessel 14. In some embodiments, the analyte nucleic acid is concentrated at least 10-fold (e.g., at least 10, 15, 20, 25, 30, 35, 40, 45, 50, 55, 60, 65, 70, 75, 80, 85, 90, 90, 100, 150, 200, 250, 300, 350, 400, 450, 500, 550, 600, 650, 700, 750, 800, 850, 900, 950, or 1000-fold or more) relative to its concentration prior to formation of ATPS 34. In some embodiments, steps facilitating separating the two phases, such as by applying a force (e.g., gravity or centrifugation) to ATPS 34, may be utilized.

In some embodiments, third phase solution 36 and fourth phase solution 38 have different volumes, and the nucleic acid fragments 26 preferentially partition into the phase having the smaller volume. In some embodiments, the fourth phase solution 38 has a volume that is 4, 5, 6, 7, 8, 9, 10, 11, 12, 13, 14, 15, 16, 17, 18, 19, 20, 30, 40, 50, or 100, or more times the volume of the third phase solution 36 of ATPS 34.

In some embodiments, the molar concentration of the first polymer or surfactant component 17 in the first phase solution 22 may be higher than the molar concentration of at least one of the second polymer or surfactant component 31 in the third phase solution 36, the second polymer or surfactant component in the fourth phase solution 38, and the second polymer or surfactant component 31 in the second ATPS 34.

In some embodiments, the mass concentration of the first polymer or surfactant component 17 in the first phase solution 22 is higher than the mass concentration of at least one of the second polymer or surfactant component(s) 31 in the third phase solution 36, the fourth phase solution 38, or in the entire second ATPS 34.

In some embodiments, the second polymer or surfactant component(s) 31 comprising the fourth phase solution 38 include one or more phase forming dissolved salt, surfactant or polymer component such as described above, and combinations thereof.

In some embodiments, the second phase solution 24 exerts weaker excluded volume interactions and exerts more favorable electrostatic interactions, upon the target nucleic acid fragments 26 than the fourth phase solution 38.

In some embodiments, the first phase solution 22 exerts stronger excluded volume interactions upon the target nucleic acid fragments 26 than the third phase solution 36, and less favorable electrostatic interactions than the fourth phase solution 38.

In some embodiments, the second phase solution 22 exerts hydrophilic/hydrophobic interactions more favorable to partitioning of the target nucleic acid fragments 26 into said second phase 22 than exerted by the fourth phase solution 38. In some embodiments, the first phase solution 22 exerts hydrophilic/hydrophobic interactions less favorable to partitioning of the target nucleic acid fragments 26 into said first phase solution 22 than exerted by the third phase solution 36. The relationship of the chemical potential of the target nucleic acids 26 in each phase immediately after mixing and before reaching equilibrium is such that μ¹_{DNA}/μ²_{DNA}>μ³_{DNA}/μ⁴_{DNA}.

Recovery of Analyte

In recovery step 40, the concentrated target nucleic acid fragments 26 may be recovered from the third phase solution 36. FIG. 1 illustrates one optional method (steps 40a through 40c) for recovering the fragments 26, wherein the third phase solution 36 may be combined in step 40a with size fractionation components 42, with or without centrifugation, in order to precipitate undesired nucleic acids (i.e., larger than the target fragment size.) A supernatant 44 including the target fragments 26 may, in step 40b, be transferred and combined with precipitation components 46, with or without centrifugation, in order to isolate and desalinate the target nucleic acid fragments 26 from the solution as pellet 50 formed at the bottom of vessel 48. In step 40c, supernatant 44 may be removed and pellet 50 comprised of target nucleic fragments 26 may be resuspended.

In some embodiments, the size fractionation components 42 may include one or more salt, polymer and/or surfactant, and combinations thereof. Suitable polymers may include, but are not limited to, polyethylene glycol, polypropylene glycol, dextran, polyalkylene glycols, such as hydrophobically modified polyalkylene glycols, poly(oxyalkylene)polymers, poly(oxyalkylene)copolymers, such as hydrophobically modified poly(oxyalkylene)copolymers, polyvinyl pyrrolidone, polyvinyl alcohol, polyvinyl caprolactam, polyvinyl methylether, alkoxylated surfactants, alkoxylated starches, alkoxylated cellulose, alkyl hydroxyalkyl cellulose, silicone-modified polyethers, and poly N-isopropylacrylamide and copolymers thereof.

Suitable surfactants may include, but are not limited to, Triton-X, Triton-114, Igepal CA-630 and Nonidet P-40, anionic surfactants such as carboxylates, sulphonates, petroleum sulphonates, alkylbenzenesulphonates, naphthalenesulphonates, olefin sulphonates, alkyl sulphates, sulphates, sulphated natural oils & fats, sulphated esters, sulphated alkanolamides, alkylphenols, ethoxylated & sulphated, nonionic surfactants such as ethoxylated aliphatic alcohol, polyoxyethylene surfactants, carboxylic esters, polyethylene glycol esters, anhydrosorbitol ester, glycol esters of fatty acids, carboxylic amides, monoalkanolamine condensates, polyoxyethylene fatty acid amides, cationic surfactants such as quaternary ammonium salts, amines with amide linkages, polyoxyethylene alkyl & alicyclic amines, n,n,n′,n′ tetrakis substituted ethylenediamines, 2-alkyl 1-hydroxethyl 2-imidazolines, and amphoteric surfactants such as n-coco 3-aminopropionic acid/sodium salt, n-tallow 3-iminodipropionate, disodium salt, n-carboxymethyl n dimethyl n-9 octadecenyl ammonium hydroxide, n-cocoamidethyl n hydroxyethylglycine.

Suitable salts may include, but are not limited to, kosmotropic salts, chaotropic salts, inorganic salts containing cations such as straight or branched trimethyl ammonium, triethyl ammonium, tripropyl ammonium, tributyl ammonium, tetramethyl ammonium, tetraethyl ammonium, tetrapropyl ammonium and tetrabutyl ammonium, and anions such as phosphates, sulphate, nitrate, chloride and hydrogen carbonate, NaCl, Na₃PO₄, K₃PO₄, Na₂SO₄, potassium citrate, (NH₄)₂SO₄, sodium citrate, sodium acetate, ammonium acetate, magnesium salt, lithium salt, sodium salt, potassium salt, cesium salt, zinc salt, aluminum salt, bromide salt, iodide salt, fluoride salt, carbonate salt, sulfate salt, citrate salt, carboxylate salt, borate salt, phosphate salt, potassium phosphate, ammonium sulfate, and combinations thereof.

In some embodiments, the molar concentration of the size fractionating component(s) 42 in the supernatant is less than the molar concentration of the first polymer or surfactant component 17 in the first phase solution 22, which in turn has a higher molar concentration than the molar concentration of the second polymer or surfactant component 31 in the third phase solution 36.

In some embodiments, the mass concentration of the at least one size fractionating component 42 in the supernatant 44 is less than the mass concentration of the first polymer or surfactant component 17 in the first phase solution 22, which in turn has a mass concentration higher than the mass concentration of the second polymer or surfactant component 31 in the third phase solution 36.

Suitable precipitation components 46 may include those known in the art, such as isopropanol, polycationic polymer salt(s), compaction agent(s) and/or a glycol.

### EXAMPLES

The present invention will be described in more detail with reference to the following examples. One skilled in the art will readily appreciate that the examples provided are merely for illustrative purposes and are not meant to limit the scope of the invention which is defined by the claims following thereafter. All references given below and elsewhere in the present application are hereby included by reference.

### Example One—Isolation of Nucleic Acids

A set of experiments were performed to observe the effect of using various concentrations of combinations of different-sized polymers on nucleic acid purification from contaminants by partitioning between top and bottom phases of an isolating polymer-salt ATPS as described above. When a biological sample is lysed, protein, RNA, genomic DNA, cells and cell debris are released. Centrifugation of a lysed sample can remove some of these contaminants, but many remain. For the purpose of some of the isolation step experiments, a preparation of 1 mL of 10% (w/v) BSA solution and different types of DNA ladder obtained from ThermoFisher Scientific or New England BioLabs was mixed in centrifuge tubes with phase forming isolating components at room temperature (15-30° C.), varied pH at 7 to 11, and centrifuged for varied duration and speed.

Glycol polymers obtained from Sigma Aldrich having molecular weights between 400 and 1200 (in 30 wt % water solution) were combined in a range from 40 uL to 80 uL in water in relative proportions along with 67 μL of phase forming salt, prepared in 30 wt % water solution to form the isolating ATPS.

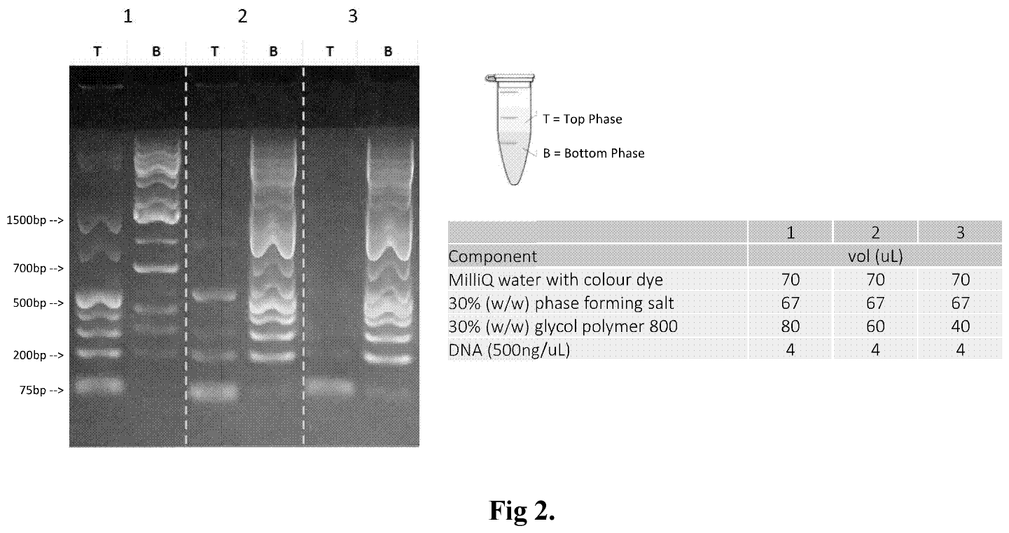

The top and bottom phases from the ATPSs were qualitatively analyzed on an agarose gel (FIG. 2). The agarose gel was run in a blueGel electrophoresis system unit from miniPCR. A 1% (w/v) agarose (Fisher BioReagents) gel containing 0.01% (v/v) GelGreen Nucleic Acid Stain (Biotium) was run for 40 min at 48V. The gel was then analyzed and photographed under UV light. From the agarose gel electrophoresis, it can be seen that the large-sized DNA fragment is to a high degree partitioned to the salt-rich bottom phase. The gel image and table illustrate the effect that changing the percentage of different-sized polymer isolating components has on nucleic acids partitioning into a polymer-rich top phase, while larger undesired DNA fragments partitioned into the salt-rich bottom phase.

Many combinations of polymers, polypropylene glycols with molecular weight ranging from 400 to 35000, polyethylene glycols with molecular weight ranging from 200 to 35000, sodium poly(acrylate) with molecular weight ranging from 8000 to 240000, Dextran with molecular weight ranging from 6000 to 65000, salts (phase-forming and non-phase forming), 0.1M to 5M sodium chloride solution, sodium sulphate solution, ammonium sulphate solution, 30 to 65 wt % potassium phosphate dibasic, 20 to 50 wt % potassium phosphate monobasic and surfactants, Triton X-100, Triton X-114, and conditions (e.g., pH, temperature) were prepared and screened by vortex mixing followed by centrifugation at various spinning time and speed for their ability to separate into thermodynamically stable isolating ATPS in optimizing for yield, stability, partitioning, compatibility with subsequent method steps and downstream PCR. In preferred embodiments, higher recovery of nucleic acids was achievable in ATPS comprised of polymers and salts, though other phase forming isolation components are suitable. The molecular weights of the polymers ranged from 200 to 240000 in concentrations of 0.1 to 40 and salts from 0.1 and 40. It was observed that generally a pH of 7 to 8 and temperatures of 20 to 30° C. led to more stable ATPS and relatively higher nucleic acid recovery. The steps in density between phases were also tuned through the addition of co-solutes.

### Example Two—Selective Isolation and Concentration of Short Nucleic Acid Fragments Using Sequential ATPS

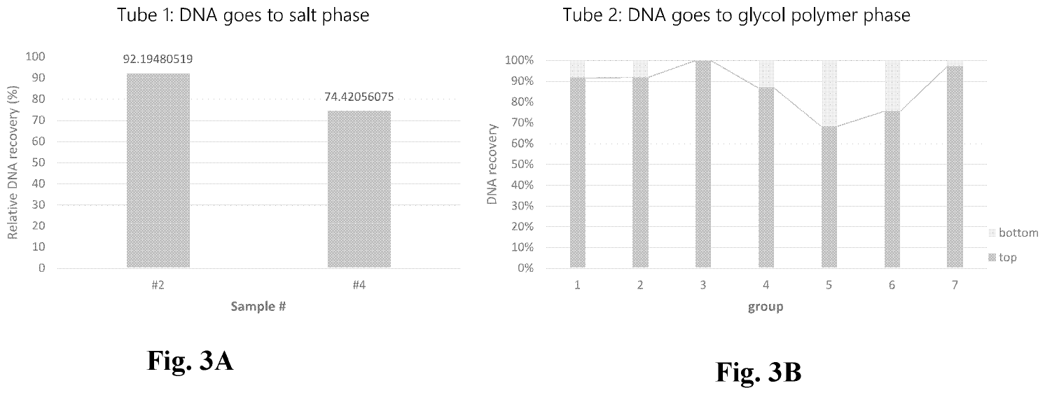

A 500 ng GeneRuler Low Range DNA Ladder from ThermoFisher Scientific was spiked into 500 μL 89 mg/mL BSA (Sigma Aldrich) solution, and after equilibrating to RT (24° C.) then mixed in a tube with an isolating components solution composed of 22.5% (v/v) glycol polymer 200MW and 18% (v/v) phase forming salt at a pH of approximately 7-9. After vortexing thoroughly, the tube was spun for 120 seconds at 7000 rcf to form a first, isolating ATPS. Contaminants partitioned into the polymer-rich top phase, while most nucleic acids at a certain size range partitioned to the salt-rich bottom phase at a volume ratio of top:bottom phase at 5:1. Among the tested formulations, the percentage of nucleic acid recovered in the bottom phase was measured and found to be, in a preferred embodiments, above 92% (run 2, FIG. 3A).

In order to concentrate the nucleic acid fragments into a smaller volume, the nucleic acid rich bottom phase was extracted from the isolation ATPS vessel and mixed with various combinations of phase-forming concentrating components, including glycol polymer 600MW (Sigma Aldrich) at a final concentration of 9.5% (v/v) and a phase forming salt at a final concentration of 25.8% (v/v), and the content is at a pH of approximately 7-9. After vortexing thoroughly, the tube was spun for 120 seconds at 7000 rcf to form a second, concentrating ATPS. Increasing the concentration of phase-forming salt results in a larger bottom phase, while the nucleic acids partition to the smaller top phase, which is freely adjustable to maximize nucleic acids concentration or partition mainly by varying the glycol polymer content

The bottom phase from the first ATPS was transferred via micropipette to a microcentrifuge tube containing the abovementioned second ATPS. Contaminants partitioned into the salt-rich bottom phase, while nucleic acids at a certain size range partitioned to and concentrated in the polymer-rich top phase, which had a volume of between ⅓ and 1/20 of the volume of the salt-rich bottom phase. In different runs, the percentage of nucleic acid recovered in the top phase was measured and found to be, in preferred embodiments over 90% and more preferably close to 100% (FIG. 3B).

### Example Three—Recovery of Isolated and Concentrate Nucleic Acids

Numerous screening experiments were conducted to develop suitable parameters for recovering nucleic acids of desired fragment sizes from solutions such as the isolated and concentrated top phase of concentrating ATPS such as described above. In some embodiments, size fractionation and salt precipitation processes were employed, wherein purified, concentrated nucleic acid solutions, Thermo GR1 kb+ and GRL, MA sizes from 25 bp to 20 kbp were first mixed by vortexing, then centrifugation at 10k rcf for 15 min, RT with various concentrations, ranging from 1-12%, of glycol polymers with MW 15000 and non-phase forming salt(s) at concentration range of 0.1M to 1.8M. Polymer/salt condensation caused nucleic acids above desired cut off sizes, which is freely adjustable above 100 bp, to precipitate to pellets at the bottom of the mixing vessel, the bottom tip of a microcentrifuge tube while a supernatant was formed including nucleic acids fragments smaller than the selected size. The supernatant, with final volume depending on formulation, was then transferred via micropipette and mixed with precipitation components including polyethyleneimine, spermine, quatroquat, NaI, spermine-HCl, trivalent spermidine and salt to neutralize the pH to around 8-10. The tubes were then spun at 20 krcf for 2 minutes in order to isolate and desalinate the target nucleic acid fragments as a pellet.

Cut-off sizes were found to be precisely tunable with numerous factors, including glycol polymers with different MW of 2k to 20k at 1-12% (v/v) final concentration, non-phase forming salt concentration at 0.1M to 1.8M, incubation temperature from 0° C. to 30° C., incubation time from 5 to 20 minutes, centrifugation time and speed at a combination of 10 krcf 15 minutes to 20 krcf 1 minute. Different co-precipitants were studied, and several were found helpful in removing large-sized DNA bands as well as stabilizing the selected cut off.

In one example embodiment, samples 1-9 were prepared and analyzed to evaluate the effects of changing different parameters in polymer/salt DNA precipitation system by several nucleic acid recovery experiments. The prepared nucleic acid mixture sample solutions (Thermo GR1 kb+ and GRL, MA sizes from 25 bp to 20 kbp) spiked with plasma were mixed in a tube with the first ATPS component and incubated for about 15 min at about 37° C. After vortexing thoroughly, the tubes were centrifuged for about 1 minute at 7000 rcf to form a first, isolating ATPS. Each of the first bottom phase from the first ATPS was then transferred to a new tube containing the second ATPS component. After vortexing thoroughly, the tubes were centrifuged for about 1 minute at 7000 rcf to form a second, concentrating ATPS. Then, each of the second top phase with a reaction volume (uL) from the second ATPS was transferred to a new tube, respectively. Fractionation component containing glycol polymer P10, salt S17 and an additive EDTA were added to the second top phase from the second ATPS to form a fractionation mixture. After vortexing thoroughly, the tube was incubated at room temperature for 10 min (or 30 min for sample 9) and spun for 10 minutes at about 16,000 rcf. For each sample, a pellet “P” and a supernatant “S” were obtained. The supernatants “S” were transferred to new tubes and mixed with precipitation components as described and salt to neutralize the pH to around 8-10. The tubes were then spun at 20 krcf for 2 minutes in order to isolate and desalinate the target nucleic acid fragments as a pellet, which was further resuspended in a suitable buffer for analysis. The pellets “P” obtained were resuspended in buffer for analysis. Tables 1-3 shows the details of the first ATPS component and the second ATPS component and the experimental conditions employed in the nucleic acid recovery experiments in this example embodiment.

TABLE 1
Composition of Example first ATPS component in the nucleic
acid recovery experiments.

| First ATPS component | Volume (uL) |
|---|---|
| Sample | 1000 |
| 65% (w/w) non-phasic salt | 241.6 |
| 100% glycol polymer with MW 600 | 196.2 |

TABLE 2
Composition of Example second ATPS component in the
nucleic acid recovery experiments.

| Second ATPS component | Volume (uL) |
|---|---|
| Bottom phase from the First ATPS | About 800 uL |
| 65% (w/w) non-phasic salt | 178.6 |
| 100% glycol polymer with MW 200 | 60 |

TABLE 3
Detailed experimental conditions employed in the nucleic acid recovery experiments.

| | | Decrease | Increase | Decrease | Increase | Decrease | Increase | Decrease | Increase |
|---|---|---|---|---|---|---|---|---|---|
| | Control | Polymer | Polymer | Salt | Salt | EDTA | EDTA | DNA | incubation |
| Sample | 1 | 2 | 3 | 4 | 5 | 6 | 7 | 8 | 9 |
| Reaction | 80.7 | 69.7 | 99.7 | 76.6 | 88.6 | 79.9 | 82.3 | 80.7 | 80.7 |
| volume | | | | | | | | | |
| (uL) | | | | | | | | | |
| Amount of | 500 | | | | | | | 100 | |
| Total DNA | | | | | | | | | |
| spike in | | | | | | | | | |
| (ng) | | | | | | | | | |
| Working | 26 | 14.3 | 40.1 | 27.4 | 23.7 | 26.3 | 25.5 | 26 | 26 |
| Concentra- | | | | | | | | | |
| tion of | | | | | | | | | |
| glycol | | | | | | | | | |
| polymer | | | | | | | | | |
| P10 (% | | | | | | | | | |
| w/v) | | | | | | | | | |
| Working | 0.5 | 0.58 | 0.41 | 0.26 | 0.9 | 0.51 | 0.49 | 0.5 | 0.5 |
| Concentra- | | | | | | | | | |
| tion of salt | | | | | | | | | |
| S17 (M) | | | | | | | | | |
| Working | 0.01 | 0.01 | 0.01 | 0.01 | 0.01 | 0.005 | 0.02 | 0.01 | 0.01 |
| Concentra- | | | | | | | | | |
| tion of | | | | | | | | | |
| EDTA (M) | | | | | | | | | |
| Incuba- | RT, 10 min | | | | | | | | RT, 30 |
| tion | | | | | | | | | min |
| condition | | | | | | | | | |

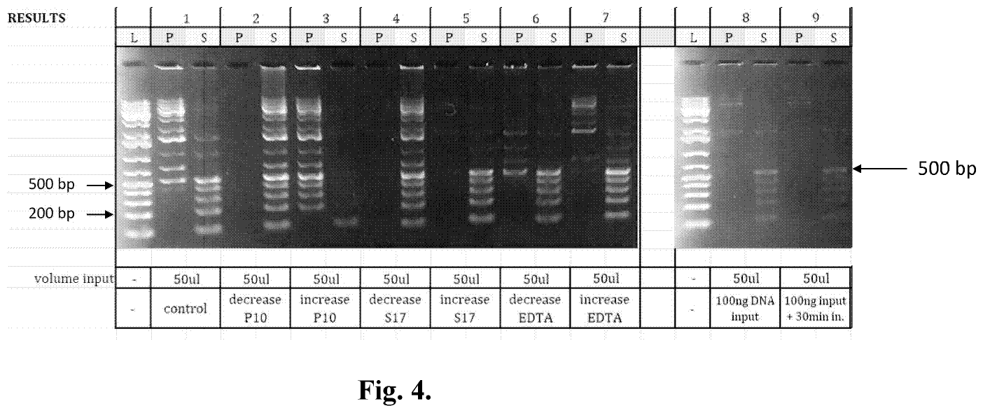

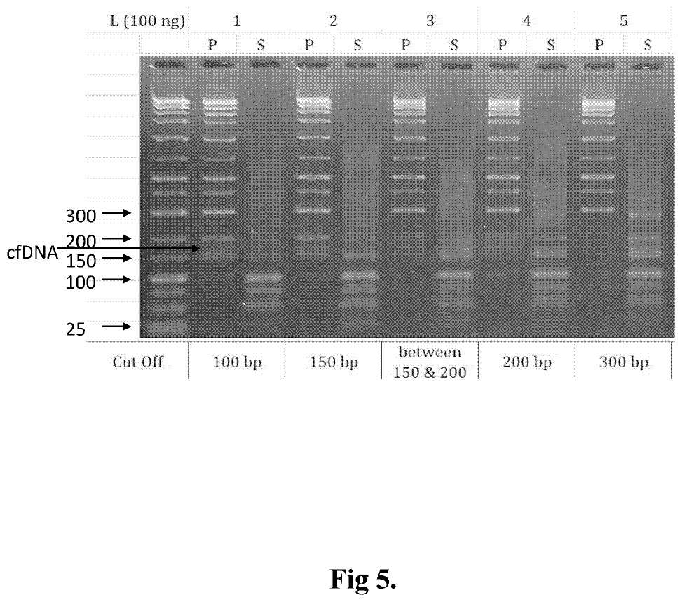

FIG. 4 shows pellet and supernatant gel image results for several nucleic acid recovery experiments. “L” denotes the DNA ladder (Thermo GR1 kb+ and GRL, MA sizes from 25 bp to 20 kbp) directly loaded into the gel for reference, “P” denotes the DNA recovered in the pellet and “S” denotes the DNA recovered in the supernatant obtained in the final step. These demonstrate how varying several precipitation components (polymers, salts, co-precipitants) affect size cut-off. Results showed that a decrease in the polymer concentration in the fractionation component from 26.0% to 14.3% would change the size cut-off from preferably less and 500 bp (sample 1) to no cut off, i.e., all sizes of DNA fragments will be retained in the supernatant (sample 2), while an increase in the polymer concentration in the fractionation component from 26.0% to 40.1% would change the size cut-off from preferably less and 500 bp (sample 1) to 200 bp, i.e. only around 200 bp or less DNA fragments will be retained in the supernatant (sample 3). Results showed that a decrease in the salt concentration from 0.5% to 0.26% in the fractionation component would cause an adverse effect to the DNA precipitation in the pellet and DNA fragment of all sizes will be retained in the supernatant (sample 4). An increase in the salt concentration in the fractionation component from 0.5% to 0.9% would also cause an adverse effect to the DNA precipitation in the pellet (sample 5), but the DNA fragments larger than 500 bp will not be retained the supernatant. Results in sample 6 (with decreased EDTA concentration) and in sample 7 (with increased EDTA concentration) show that variation of the concentration of EDTA does not contribute to change in size cut-off. Results further show that a decrease of the amount of total DNA spiked from 500 ng to 100 ng (sample 8) significantly decreases the final yield (i.e., the DNA band intensity is lighter) but does not affect the size cut-off Longer incubation time (an increase from 10 minutes to 30 minutes) does not increase the final yield (sample 9). In general, varying the concentration of the polymer(s) used will change size cut-off from 500 bp to 200 bp or from 500 bp to no cut-off as shown in the gel image, while changing the concentration of the salt employed will affect precipitation ability, removal of large-sized DNA fragments as the concentration of salt increase. Double precipitation methods were tried and found to generate consistent results, but the yields of target size nucleic acid fragments decreased. It was also discovered that lower initial nucleic acid concentrations significantly decreased the yield and purity of the resulting nucleic product, but not the size cut-off. This observation can be solved by adjusting the concentration of each component added (FIG. 5).

In one example embodiment, samples 1-5 were prepared and analysed to evaluate the robustness of the recovery system experiments. Tables 1-2 show the details of the first ATPS component and the second ATPS component and Table 4 shows the experimental conditions employed in the nucleic acid recovery experiments in this example embodiment.

TABLE 4
Detailed experimental conditions employed in the robust ability of recovery
system experiments.

| | Cut-off target | | | | |
|---|---|---|---|---|---|
| | 100 bp | 150 bp | 150-200 bp | 200 bp | 300 bp |
| | Sample | | | | |
| | 1 | 2 | 3 | 4 | 5 |
| Reaction volume | 391.1 uL | | | | |
| Amount of DNA spike-in | 500 ng | | | | |
| Volume of reaction input | 250 uL | | | | |
| (Top phase from 2nd ATPS*) | | | | | |
| Working Concentration of | 14.5 | 13.4 | 12.4 | 11.3 | 10.2 |
| glycol polymer P10(% w/v) | | | | | |
| Working Concentration of | 0.78 | 0.81 | 0.84 | 0.86 | 0.88 |
| Salt S17 (M) | | | | | |
| Working concentration of | 0.0049 | | | | |
| additive EDTA (M) | | | | | |
| Amount of additive glycogen | 0.15 | | | | |
| added (mg) | | | | | |

| | |
|---|---|
| Incubation condition | RT, 10 min |
| Centrifugation condition | 20000 rcf for 5 min at RT |

FIG. 5 illustrates gel images demonstrating the robust ability of recovery system embodiments to fine tune with relative ease through design parameters adjustments the nucleic acid fragment size cut-off within increments of 50 bp between 100 bp and 300 bp. Total amount of 100 ng of NEB 1 kb ladder and Thermo GRL were pre-spiked into plasma samples and the samples were run through the present invention.

In the example embodiment, “cfDNA” denotes an example target DNA size of 150-200 bp, for example around 170 bp. Results in samples 1-2 showed that most cfDNA would be precipitated out to the pellet, while in samples 3-5, cfDNA would retain in the supernatant. By only varying the concentrations of the components in the polymer/non-phase forming salt precipitation step, with glycol polymer 15000 at 1-12% (v/v), non-phase forming salt concentration at 0.1M to 1.8M, the mentioned desirable cut-off range can be achieved. This range was selected only for exemplary purposes and its relation to small sizes encountered with analyses of cfDNA.

### Example Four—Comparison with the Qiagen QIAamp MinElute ccfDNA Mini Kit

In this example embodiment, the ATPS system was compared with a commercially available kit using the same DNA sample. Thermo GR1 kb+ and GRL, MA sizes from 25 bp to 20 kbp was spiked into plasma to form al kb ladder sample mixture for test. Same amount of 1 kb ladder sample mixture were used for the tests. For sample using Qiagen QIAamp MinElute ccfDNA Mini Kit (Qiagen Kit), the nucleic acids were prepared according to the manufacturer's instructions. For sample using ATPS system, the prepared nucleic acid mixture solution was mixed in a tube with the first ATPS component and incubated for about 15 min at about 37° C. After vortexing thoroughly, the tube was centrifuged for about 1 minute at 7000 rcf to form a first isolating ATPS. The first bottom phase from the first ATPS was then transferred to a new tube containing the second ATPS component. After vortexing thoroughly, the tube was centrifuged for about 1 minute at 7000 rcf to form a second, concentrating ATPS. Then, each of the second top phase with an reaction volume (uL) from the second ATPS was transferred to a new tube, respectively. Fractionation component containing glycol polymer P10, salt S17 and an additive EDTA were added to the second top phase from the second ATPS to form a fractionation mixture. After vortexing thoroughly, the tube was incubated at room temperature for 10 min (or 30 min for sample 9) and spun for 10 minutes at about 16,000 rcf. For each sample, a pellet “P” and a supernatant “S” were obtained. The supernatants “S” were transferred to new tubes and mixed with precipitation components. The tubes were then centrifuged at 20 krcf for 2 minutes in order to isolate and desalinate the target nucleic acid fragments as a pellet, which is further resuspended in a suitable buffer for analysis. The pellets “P” obtained were resuspended in buffer for analysis. Tables 1-2 show the details of the first ATPS component and the second ATPS component and Table 5 shows the experimental conditions employed in the nucleic acid recovery experiments in this example embodiment. The recovered DNA samples from the ATPS system and the commercial kit for comparison were analyzed by qPCR (Roche).

TABLE 5
Detailed experimental conditions employed in ATPS system.

| ATPS System Sample | Condition |
|---|---|
| Reaction volume | 383.6 uL |
| Volume of reaction input | 250 uL |
| (Top phase from 2nd ATPS) | |
| Working Concentration of glycol | 11.1% (w/v) |
| polymer 6000 | |
| Working Concentration of Salt | 0.97 M |
| Working concentration of EDTA | 0.02 M |
| Incubation condition | RT, 15 min |
| Centrifugation condition | RT, 10 min, 16000 rcf |

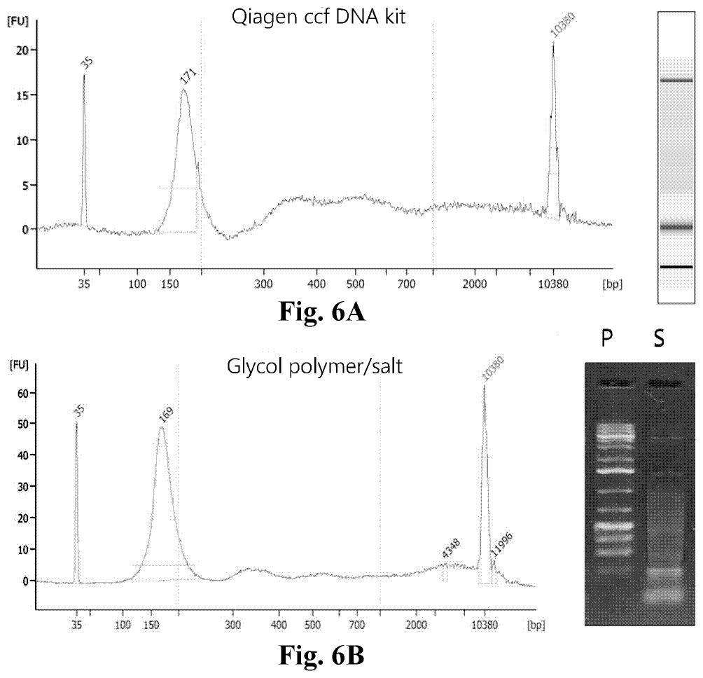

As shown in FIG. 6, when using the present invention, close to 100%, analyzed through qPCR (Roche), of nucleic acids, 130 bp spike-in oligonucleotide, were extracted from plasma sample by the sequential ATPS method, while the Qiagen kit extracted about 50% of such nucleic acids. Therefore, the present invention showed an improved performance compared to the QIAamp MinElute ccfDNA Mini Kit.

### Example Five—Selective Isolation and Concentration of Target Nucleic Acid Fragments Using GlycolPolymer/Sodium Poly(Acrylate) and Glycol Polymer/Triton ATPS System

In this example embodiment, samples were prepared and analyzed to evaluate an example ATPS system by several nucleic acid recovery experiments. The prepared nucleic acid mixture sample solutions (Thermo GR1 kb+ and GRL, MA sizes from 25 bp to 20 kbp) spiked with plasma were mixed in a tube with the first ATPS component (glycol polymer/sodium poly(acrylate) and incubated for about 15 min at about 37° C. After vortexing thoroughly, the tubes were centrifuged for about 1 minute at 7000 rcf to form a first, isolating ATPS. Each of the bottom phase from the first ATPS was then transferred to a new tube containing the second ATPS component (glycol polymer/salt/Triton with concentrations of 0, 10, 20 and 30%). After vortexing thoroughly, the tubes were centrifuged for about 1 minute at 7000 rcf to form a second, concentrating ATPS. The top (T) and bottom (B) phases were transferred to new tubes and mixed with precipitation components and salt. The tubes were then centrifuged at 20 krcf for 2 minutes in order to isolate and desalinate the target nucleic acid fragments as a pellet, which is further resuspended in a suitable buffer for analysis. Tables 6-7 shows the details of the first ATPS component and the second ATPS component in this example embodiment.

TABLE 6
Composition of Example first ATPS component in the nucleic
acid recovery experiments.

| First ATPS component | Volume (uL) |
|---|---|
| Sample | 1000 |
| 20% non-phasic salt | 10 |
| 30% glycol polymer MW10,000/10% Sodium | 400 |
| Poly(acrylate) | |

TABLE 7
Composition of Example second ATPS component in the nucleic
acid recovery experiments.

| Second ATPS component | Volume (uL) |
|---|---|
| Bottom phase from the First ATPS | About 800 uL |
| 20% non-phasic salt | 10 |
| 22% glycol polymer MW4,000/Triton MW | 300 |
| 625 | |

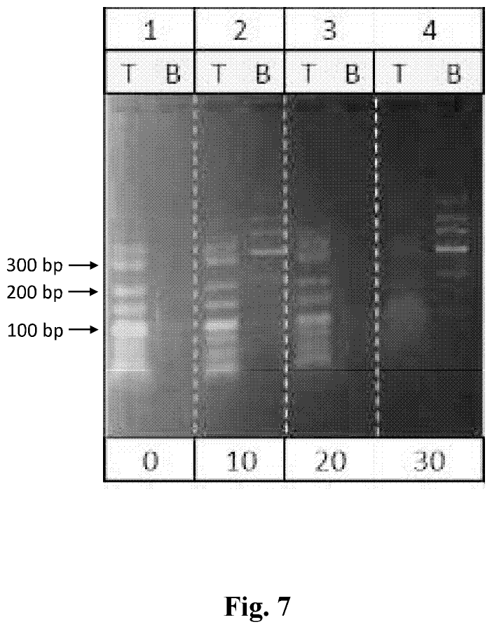

FIG. 7 shows top (T) and bottom (B) phase gel image results for the nucleic acid recovery experiments. These demonstrate how varying several precipitation components (polymers, salts, co-precipitants) affect size cut-off. Results showed that most proteins and contaminants will be partitioned to the first bottom phase in first ATPS system (the glycol polymer/sakt/Sodium Poly(acrylate)) and most of the nucleic acids would be retained in the first top phase. FIG. 7 shows that an increase in concentration of glycol polymer/salt/Triton in the second ATPS system would change the nucleic acid size cut-off selection from no cut off to around 300 bp to be retained in the second top (T) phase, and the larger undesired DNA would be partitioned to the second bottom (B) phase. In general, varying the concentration of the glycol polymer/Triton used will change the desired nucleic acid size cut-off in the second top phase as shown in the gel image.

### Example Six—Selective Isolation and Concentration of Target Nucleic Acid Fragments Using ATPS System with Different Salt Concentrations

In this example embodiment, samples were prepared and analyzed to evaluate various concentrations of sodium sulphate by several DNA and protein recovery experiments. A nucleic acid mixture sample solution (500 ng/uL) spiked with protein of 89.3 mg/ml were prepared and mixed in a tube with the first ATPS component with different concentrations of sodium sulphate and incubated for about 15 min at about 37° C. After vortexing thoroughly, the tubes were centrifuged for about 1 minute at 7000 rcf to form a first, isolating ATPS. Each of the bottom phase from the first ATPS was then transferred to a new tube containing the second ATPS component. After vortexing thoroughly, the tubes were centrifuged for about 1 minute at 7000 rcf to form a second, concentrating ATPS. The top (T) and bottom (B) phases were transferred to new tubes and mixed with precipitation components and salt. The tubes were then centrifuged at 20 krcf for 2 minutes in order to isolate and desalinate the target nucleic acid fragments as a pellet, which is further resuspended in a suitable buffer for analysis. The DNA recovery % and the protein recovery % in the top and bottom phases were determined. Table 8 shows the experimental details in this example embodiment.

TABLE 8
Composition of experimental details in the nucleic acid and protein recovery
experiments. ″Top 1″ denotes the top phase of sample 1; ″bottom 1″ denotes the bottom
phase of sample 1; ″top 2″ denotes the top phase of sample 2; ″bottom 2″ denotes the bottom
phase of sample 2; ″top 3″ denotes the top phase of sample 3; ″bottom 3″ denotes the bottom
phase of sample 3.

| | Sample | | |
|---|---|---|---|
| | 1 | 2 | 3 |
| Component volume | vol (uL) | | |
| Water | 20 | 0 | 0 |
| Sodium Sulphate | 400 | 250.5 | 275.5 |
| Glycol polymerl | — | 57.5 | 52.5 |
| glycol polymer 2 | 78 | — | — |
| DNA spike (500 ng/uL) | 2 | 2 | 2 |
| Protein spike (89.3 mg/ml) | 200 | 200 | 200 |

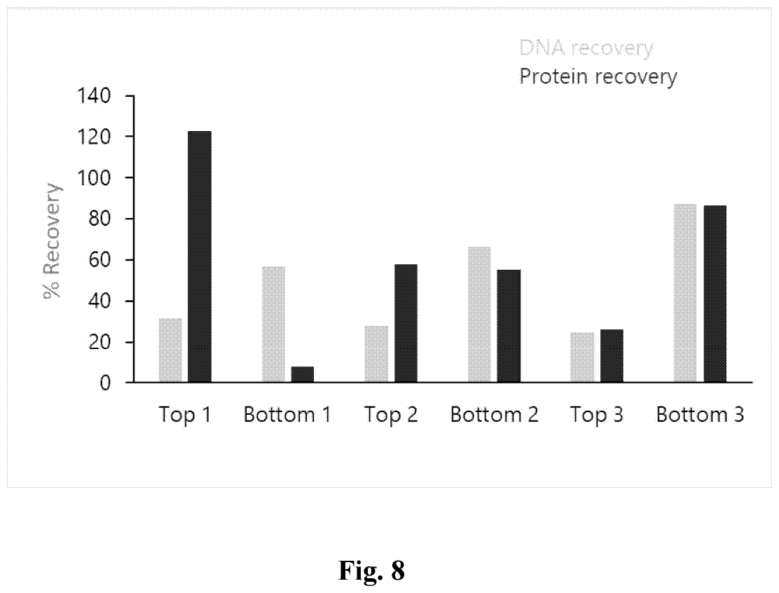

FIG. 8 shows the DNA and protein recovery results for the nucleic acid and protein recovery experiments. Results showed that an increase in the sodium sulphate salt concentration from 0.1% to 0.8% would cause higher DNA recovery from around 60% to 85% (samples 2 and 3) in the bottom phase.

### Example Seven—Selective Isolation and Concentration of Target Nucleic Acid Fragments Using Multiple Triton ATPS Systems

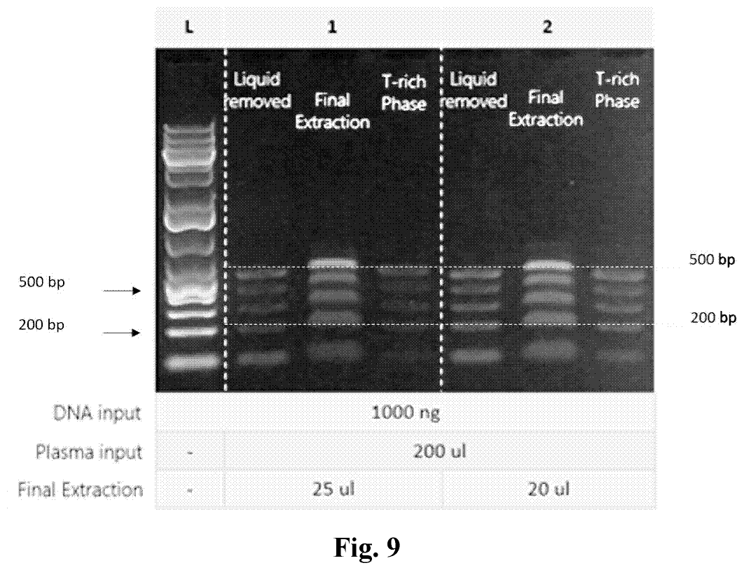

In this example embodiment, samples were prepared and analyzed to evaluate various concentrations of sodium sulphate by several DNA and protein recovery experiments. A nucleic acid mixture sample solution (total 1000 ng) spiked with plasma protein 200 ul were prepared and mixed in a tube with the first ATPS component to form the first ATPS system (water/Triton ATPS system) and incubated for about 15 min at about 37° C. After vortexing thoroughly, the tubes were centrifuged for about 1 minute at 7000 rcf to form a first, isolating ATPS. Each of the bottom phase from the first ATPS was then transferred to a new tube containing precipitation component. After vortexing thoroughly, the tubes were centrifuged for about 1 minute at 7000 rcf to form nucleic acid pellet in the salt precipitation system. The supernatant containing the contaminants were removed. Then, the pellet was added with the second ATPS component. After vortexing thoroughly, the tubes were centrifuged for about 1 minute at 7000 rcf to form a second, isolating ATPS (glycol polymer/salt ATPS system). The second upper phase containing smaller sized nucleic acids were transferred to new tubes and mixed with the third ATPS component to form the third ATPS system (glycol polymer/Triton system). After vortexing thoroughly, the tubes were centrifuged for about 1 minute at 7000 rcf to form a third ATPS (glycol polymer/Triton ATPS system) in the upper white suspension. Then, water was added to resuspend the white suspension to form the water/Triton system with an upper Triton-poor phase and lower Triton-rich phase. The final extraction volumes used in samples 1 and 2 are 25 ul and 20 ul, respectively. The upper Triton-poor phase is the final product containing clean, target nucleic acids. The liquid removed, the final extraction (i.e., Triton-poor phase) and the Triton-rich phase were analysed by gel electrophoresis. FIG. 9 shows the liquid removed, final extraction and T-rich phase gel image results. “L” denotes the DNA ladder (Thermo GR1 kb+ and GRL, MA sizes from 25 bp to 20 kbp) directly loaded into the gel for reference, Results showed that the targeted nucleic acids are present in all fractions, indicating some loss of targeted nucleic acids in the series of extraction steps. Results also showed that the final extraction contains significant amount of target nucleic acids. The target 75 bp band was retained in the final extraction and the spiked proteins are removed. Both the quality and quantity of the target nucleic acids in the final extraction were sufficient for gel electrophoresis analysis. In summary, the multiple Triton ATPS systems are efficient in isolating, purifying and concentrating target nucleic acids for downstream applications.

### Example Eight—Recovery of Isolated and Concentrate Nucleic Acid Using Polymers with Different Molecular Weights

In this example embodiment, samples were prepared and analyzed to evaluate the effects of changing different molecular weight of a polymer in the second ATPS system to the nucleic acid recovery. The prepared nucleic acid mixture of about 50 ng sample solutions spiked with plasma were mixed in a tube with the same first ATPS component and incubated for about 15 min at about 37° C. After vortexing thoroughly, the tubes were centrifuged for about 1 minute at 7000 rcf to form a first, isolating ATPS. Each of the first bottom phase from the first ATPS was then transferred to a new tube containing the second ATPS component, either containing low molecular weight or medium molecular weight polymer. The polymer and the salt in the second ATPS component in all samples were kept at the same concentrations of 7% and 35%, respectively. After vortexing thoroughly, the tubes were centrifuged for about 1 minute at 7000 rcf to form a second, concentrating ATPS. Then, each of the second top phase with a reaction volume (uL) from the second ATPS was transferred to a new tube, respectively. The supernatants were transferred to new tubes and mixed with precipitation components and salt to neutralize the pH to around 8-10. The tubes were then spun at 20 krcf for 2 minutes in order to isolate and desalinate the target nucleic acid fragments as a pellet, which is further resuspended in a suitable buffer 15 ul for analysis. The nucleic acid yields of the final project were determined by Qubit Fluorometric Quantification (Invitrogen) and the recovery were calculated from the yields. Table 9 shows the experimental details and the DNA yield and recovery results in this example embodiment. In this example embodiment, the molecular weight difference from “Low” to “Medium” is within 100-1000 Da.

TABLE 9
Experimental details and the DNA yield and recovery results using
polymers with different MW.

| | | | | top | | | | | | | |
|---|---|---|---|---|---|---|---|---|---|---|---|
| | | | | phase | | | DNA | DNA | | | |
| Polymer | polymer | | | volume | | | conc. | yield | % DNA | Avg. % | |
| MW | % | salt % | Replicate | (ul) | Avg. | SD | (ng/uL) | (ng) | recovery | recovery | SD |
| Low | 7 | 35 | #1 | 183.4 | 183.70 | 0.30 | 3.68 | 55.2 | 98.05 | 92.45* | 5.60 |
| | | | #2 | 184 | | | 3.26 | 48.9 | 86.86 | | |
| | | | #3 | N/A | | | 2.052 | 30.78 | 54.67 | | |
| Medium | 7 | 35 | #1 | 180.4 | 188.33 | 12.51 | 3.44 | 51.6 | 91.65 | 81.44 | 8.72 |
| | | | #2 | 178.6 | | | 2.64 | 39.6 | 70.34 | | |
| | | | #3 | 206 | | | 3.09 | 46.35 | 82.33 | | |

“N/A” denotes the transfer volume was failed to record.
*Note
that the calculation of average recovery has excluded the outlier value of 54.67%.

Table 9 showed that an increase in the polymer molecular weight from “low” to “medium” would result in a worse recovery rate, indicating that polymers with specific molecular weights may produce outstanding performances in nucleic acid recovery.

### Example Nine—Recovery of Isolated and Concentrate Nucleic Acid with Different Top Phase Volumes in the Second ATPS

In this example embodiment, samples were prepared and analyzed to evaluate the effects of changing the volume of the top phase in the second ATPS system to the recovery of nucleic acids. One nucleic acid mixture was prepared and aliquoted in the tubes with the same first ATPS component and incubated for about 15 min at about 37° C. After vortexing thoroughly, the tubes were centrifuged for about 1 minute at 7000 rcf to form a first, isolating ATPS. Each of the first bottom phase from the first ATPS was then pooled together and aliquoted to new tubes containing the second ATPS component, with different volumes of polymer (PM2) and salt (SM2) in the second ATPS component, according to Table 10. After vortexing thoroughly, the tubes were centrifuged for about 1 minute at 7000 rcf to form a second, concentrating ATPS. Then, the volume of each of the second top phase from the second ATPS was determined. Each of the second top phase was transferred to a new tube, respectively. The supernatants were transferred to new tubes and mixed with precipitation components and salt to neutralize the pH to around 8-10. The tubes were then spun at 20 krcf for 2 minutes in order to isolate and desalinate the target nucleic acid fragments as a pellet, which is further resuspended in a suitable buffer for analysis. The ability to form a clean pellet, the nucleic acid purity by the ratio of A260/A280, the final product concentration and the total yield were evaluated and determined for each sample. Table 10 shows the experimental details and the results in this example embodiment.

TABLE 10
Experimental details and the results using different volumes of top phase in the second ATPS.

| | | | Top | | | | DNA |
|---|---|---|---|---|---|---|---|
| | ATPS2 component | | Phase | | | DNA | final |
| | polymer | | volume | Clean pellet? | | conc. | yield |
| Sample | (uL) | salt (uL) | (uL) | (yes/acceptable/no) | A260/A280 | (ng/uL) | (ng) |
| 1 | 60 | 50 | 127 | yes | 1.28 | 0.069 | 1.38 |
| 2 | 100 | 20 | 220 | acceptable | 0.79 | 0.065 | 1.3 |
| 3 | 100 | 200 | 234 | acceptable | 0.88 | 0.211 | 4.22 |
| 4 | 200 | 200 | 452 | no | N/A | N/A | 0 |
| 5 | 100 | 400 | 210 | yes | 1 | 0.171 | 3.42 |
| 6 | 200 | 400 | 415 | no | N/A | N/A | 0 |
| 7 | 100 | 600 | 178 | yes | 1.3 | 0.169 | 3.38 |
| 8 | 200 | 600 | 345 | no | N/A | N/A | 0 |
| 9 | 200 | 800 | 333 | no | N/A | N/A | 0 |
| 10 | 300 | 800 | 480 | no | N/A | N/A | 0 |

“N/A” denotes not applicable because DNA pellet was failed to form.
The result of forming a clean pellet is denoted as “yes”; the result of forming a poor, loose and/or dirty pellet is denoted as “acceptable”; the result of forming no pellet is denoted as “no”.

Whilst it would be advantageous to have larger top phase volumes because it tends to minimize the loss of nucleic acid during transfer or extraction, Table 10 showed that larger top phase volumes would actually produce only acceptable (samples 2 and 3) or even no DNA pellets (samples 4, 6, 8-10). Samples with lower concentrations of salt (sample 2) produced poor, unstable pellets. Larger salt volumes (samples 5-10) produced more stable pellets and reasonable yields. To achieve an increase in the volume of top phase, the polymer concentration needs to be increased accordingly. It is found that when the polymer concentration was increased to be greater than 8.7%, it would give the top phase volumes more than 200 μL. The data in table 10 showed that the top phase volumes larger than 220 μL would result in failure of the nucleic acid extraction process. Even if the nucleic acid precipitation is successful, they produce pellets that are not clean (either strange or junk pellets) and the results also reflects in the purity of DNA (A260/A280). In summary, it is discovered that the polymer concentration and the salt concentration in the second ATPS being less than 8.7% and 1%, respectively, and the final volume of the top phase being less than 200 uL would significantly improve the yield and purity of the resulting nucleic product of the ATPS system.

### Example Ten—Recovery of Isolated and Concentrate Nucleic Acid with Different Molecular Weights of Polymers in First and Second ATPS Systems

In this example embodiment, samples were prepared and analyzed to evaluate the effects of using different molecular weights of polymers in the first and the second ATPS systems to the recovery of nucleic acids. A nucleic acid mixture sample solution was prepared and aliquoted in the tubes with first ATPS components with different polymer molecular weights (according to Table 11) and incubated for about 15 min at about 37° C. After vortexing thoroughly, the tubes were centrifuged for about 1 minute at 7000 rcf to form a first, isolating ATPS. Each of the first bottom phase from the first ATPS was transferred to new tubes containing the second ATPS components with different polymer molecular weights (according to Table 11). After vortexing thoroughly, each of the tubes were centrifuged for about 1 minute at 7000 rcf to form a second, concentrating ATPS. Each of the second top phase was transferred to a new tube, respectively. The supernatants were transferred to new tubes and mixed with precipitation components and salt to neutralize the pH to around 8-10. The tubes were then spun at 20 krcf for 2 minutes in order to isolate and desalinate the target nucleic acid fragments as a pellet, which is further resuspended in a suitable buffer with the same volume for analysis. The nucleic acid purity by the ratio of A260/A280 and the DNA concentration were determined measured by spectrophotometer (Nanodrop) for each sample. Table 11 shows the experimental details and the results in this example embodiment. In this example embodiment, the molecular weight difference from “Low” to “High” is within 200-2000 Da.

TABLE 11
Experimental details and the results using different polymer
molecular weights in the first ATPS and the second ATPS. DNA
was not obtained in Samples 3 and 4 because
no phase is formed in the first ATPS.

| | First | Second | | | |
|---|---|---|---|---|---|
| | ATPS | ATPS | | | |
| | polymer | polymer | DNA concentration (ng/uL) | | |
| Sample # | MW | MW | #1 | #2 | Average |
| 1 | High | Low | 26.4 | 21.9 | 24.2 |
| 2 | High | High | 17.0 | 17.8 | 17.4 |
| 3 | Low | Low | No phase in ATPS 1 | | |
| 4 | Low | High | | | |

Table 11 showed that the DNA concentration would have a significant decrease from around 24 ng/ul to around 17 ng/ul when a high molecular weight polymer was used instead of a lower molecular weight polymer in the second ATPS (samples 1 and 2). It also showed that if a low molecular weight polymers was used in the first ATPS, a phase would not even form in the first ATPS and thus nucleic acids would not be obtained. The results showed that the choices of molecular weight of polymer in the first and the second ATPS systems significantly affect the nucleic acid recovery in addition to the target size cut off selection. The first polymer component having a higher molecular weight than the second polymer component would significantly improve the yield and purity of the resulting nucleic product of the ATPS system.

### Example Eleven—Recovery of Isolated and Concentrate Nucleic Acid with Different Molecular Weights and Concentrations of Polymers in the Fractionation Component

In this example embodiment, samples were prepared and analyzed to evaluate the effects of using different molecular weights and different concentrations of polymers in the fractionation component to the recovery of nucleic acids. A nucleic acid mixture of about 200 ng/mL DNA ladder (Thermo GR1 kb+ and GRL, MA sizes from 25 bp to 20 kbp) sample mixture solution was prepared and aliquoted in the tubes with the same first ATPS component and incubated for about 15 min at about 37° C. After vortexing thoroughly, the tubes were centrifuged for about 1 minute at 7000 rcf to form a first, isolating ATPS. Each of the first bottom phase from the first ATPS was transferred to new tubes containing the second ATPS components with different polymer molecular weights (according to Table 11). After vortexing thoroughly, each of the tubes were centrifuged for about 1 minute at 7000 rcf to form a second, concentrating ATPS. Each of the second top phase was transferred to a new tube, respectively. Then, each of the second top phase from the second ATPS was transferred to a new tube, respectively. Fractionation components containing different molecular weights or different concentrations of polymer (please refer to Table 12) were added to the second top phase from the second ATPS to form fractionation mixtures, respectively. After vortexing thoroughly, each of the tube was incubated at room temperature for 10 min (or 30 min for sample 9) and spun for 10 minutes at about 16,000 rcf. For each sample, a pellet “P” and a supernatant “S” were obtained. The supernatants “S” were transferred to new tubes and mixed with precipitation components and salt to neutralize the pH to around 8-10. The tubes were then spun at 20 krcf for 2 minutes in order to isolate and desalinate the target nucleic acid fragments as a pellet, which is further resuspended in 10 uL suitable buffer (1× loading dye) for analysis. The pellets “P” obtained were resuspended in the same way for analysis. Table 11 shows the experimental details and the results in this example embodiment. In this example embodiment, the molecular weight difference from “Low” to “High” is within 200-2000 Da.

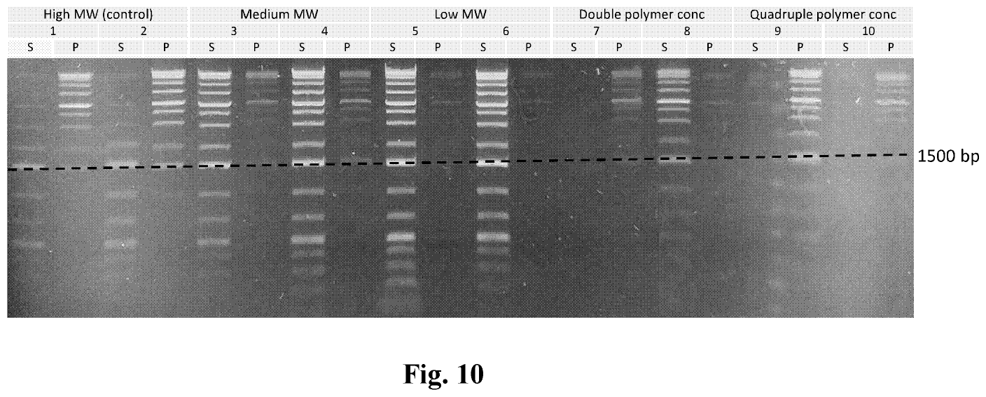

FIG. 10 shows pellet “P” and supernatant “S” gel image results for the nucleic acid recovery experiments in the example embodiment. The molecular weight difference from “Low” to “Medium” is within 100-1000 Da while the molecular weight difference from “Medium” to “High” is within 100-1000 Da. “P” denotes the DNA recovered in the pellet and “S” denotes the DNA recovered in the supernatant obtained in the final step. Dash line indicates the molecular size of about 1500 bp. Results showed that a cut-off size of below around 1500 bp would be achieved if a high MW polymer is used in the fractionation component (samples 1 & 2). If the molecular weight of the polymer changes from high to medium or even low molecular weight, the cut-off size of below around 1500 bp would not be achieved, and most of the nucleic acids with all fragment sizes tend to retain in the supernatant (samples 3-6). Results further showed that using double or quadruple concentrations of polymer would not achieve satisfactory cut-off size with reasonable yield because most of the nucleic acid with all fragment sizes would retain in the pellets (samples 7-10). In summary, high molecular weight of polymer with specific concentrations would achieve satisfactory size-cut off and nucleic acids recovery results.

The exemplary embodiments of the present invention are thus fully described. Although the description referred to particular embodiments, it will be clear to one skilled in the art that the present invention may be practiced with variation of these specific details. Hence this invention should not be construed as limited to the embodiments set forth herein.

1. A method for isolating and concentrating nucleic acids of target sizes from a fluid mixture including nucleic acids and contaminants, comprising:
combining said fluid mixture and a first aqueous two-phase system (ATPS) formed from a first phase forming polymer or surfactant component dissolved in a first phase solution, and a second phase solution, such that target nucleic acid fragments below a target size partition to said second phase solution and contaminants partition to the first phase solution;
extracting and mixing the second phase solution with a second ATPS formed from a second phase forming polymer or surfactant component dissolved in a third phase solution and a fourth phase solution, such that the target nucleic acid fragments partition to and concentrate in the third phase solution; and
recovering the concentrated target nucleic acid fragments from the third phase solution.

2. The method of claim 1, wherein the first polymer or surfactant component has a higher molecular weight than the second polymer or surfactant component.

3. The method of claim 1, wherein the molar concentration of the first polymer or surfactant component in the first phase solution is higher than the molar concentration of at least one of the second polymer or surfactant component in the third phase solution, the second polymer or surfactant component in the fourth phase solution, and the second polymer or surfactant component in the second ATPS.

4. The method of claim 1, wherein the mass concentration of the first polymer or surfactant component in the first phase solution is higher than the mass concentration of at least one of the second polymer or surfactant component in the third phase solution, the second polymer or surfactant component in the fourth phase solution, and the second polymer or surfactant component in the second ATPS.

5. The method of claim 1, wherein the second phase solution comprises at least one phase forming dissolved salt, surfactant or polymer component selected from the group consisting of dipotassium phosphate, monopotassium phosphate, kosmotropic salts, chaotropic salts, inorganic salts containing cations such as straight or branched trimethyl ammonium, triethyl ammonium, tripropyl ammonium, tributyl ammonium, tetramethyl ammonium, tetraethyl ammonium, tetrapropyl ammonium and tetrabutyl ammonium, anions such as phosphates, sulphate, nitrate, chloride and hydrogen carbonate, NaCl, Na3PO4, K3PO4, Na2SO4, potassium citrate, (NH4)2SO4, sodium citrate, sodium acetate, ammonium acetate, magnesium salt, lithium salt, sodium salt, potassium salt, cesium salt, zinc salt, aluminum salt, bromide salt, iodide salt, fluoride salt, carbonate salt, sulfate salt, citrate salt, carboxylate salt, borate salt, phosphate salt, potassium phosphate, ammonium sulfate, and combinations thereof.

6. The method of claim 1, wherein the fourth phase solution comprises at least one phase forming dissolved salt, surfactant or polymer component selected from the group consisting of dipotassium phosphate, monopotassium phosphate, kosmotropic salts, chaotropic salts, inorganic salts containing cations such as straight or branched trimethyl ammonium, triethyl ammonium, tripropyl ammonium, tributyl ammonium, tetramethyl ammonium, tetraethyl ammonium, tetrapropyl ammonium and tetrabutyl ammonium, anions such as phosphates, sulphate, nitrate, chloride and hydrogen carbonate, NaCl, Na3PO4, K3PO4, Na2SO4, potassium citrate, (NH4)2SO4, sodium citrate, sodium acetate, ammonium acetate, magnesium salt, lithium salt, sodium salt, potassium salt, cesium salt, zinc salt, aluminum salt, bromide salt, iodide salt, fluoride salt, carbonate salt, sulfate salt, citrate salt, carboxylate salt, borate salt, phosphate salt, potassium phosphate, ammonium sulfate, and combinations thereof.

7. The method of claim 1, wherein the second phase solution of the first ATPS exerts weaker excluded volume interactions upon the target nucleic acid fragments than the fourth phase solution of the second ATPS.

8. The method of claim 1, wherein the first phase solution of the first ATPS exerts stronger excluded volume interactions upon the target nucleic acid fragments than the third phase solution of the second ATPS.

9. The method of claim 1, wherein the second phase solution of the first ATPS exerts hydrophilic/hydrophobic interactions more favorable to partitioning of the target nucleic acid fragments into said second phase than the fourth phase solution of the second ATPS.

10. The method of claim 1, wherein the first phase solution of the first ATPS exerts hydrophilic/hydrophobic interactions less favorable to partitioning of the target nucleic acid fragments into said first phase solution than the third phase solution of the second ATPS.

11. The method of claim 1, wherein the second phase solution of the first ATPS exerts electrostatic interactions more favorable to partitioning of the target nucleic acid fragments into said second phase than the fourth phase solution of the second ATPS.

12. The method of claim 1, wherein the first phase solution of the first ATPS exerts electrostatic interactions less favorable to partitioning upon the target nucleic acid fragments into said first phase solution than the fourth phase solution of the second ATPS.

13. The method of claim 1, wherein recovering the concentrated target nucleic acid fragments comprises:
separating the third phase solution from the fourth phase solution;
mixing the third phase solution with at least one size fractionation component selected from a polymer, a surfactant, a salt, and combinations thereof, dissolved in an aqueous solution in order to form a supernatant comprised of concentrated target nucleic acids and a precipitated pellet of nucleic acids above the target size; and
separating the supernatant from the precipitated pellet of nucleic acids above the target size and precipitating the target nucleic acid fragments from the supernatant.

14. The method of claim 13, wherein the at least one size fractionating component comprises a polymer or surfactant component having a higher molecular weight than the first polymer or surfactant component, and the first polymer or surfactant component has a higher molecular weight than the second polymer or surfactant component.

15. The method of claim 13, wherein the molar concentration of the at least one size fractionating component in the supernatant is less than the molar concentration of the first polymer or surfactant component in the first phase solution, and the first polymer or surfactant component in the first phase solution has a higher molar concentration than the molar concentration of the second polymer or surfactant component in the third phase solution.

16. The method of claim 13, wherein the mass concentration of the at least one size fractionating component in the supernatant is less than the mass concentration of the first polymer or surfactant component in the first phase solution, and the first polymer or surfactant component in the first phase solution has a mass concentration higher than the mass concentration of the second polymer or surfactant component in the third phase solution.

17. The method of claim 13, wherein the at least one size fractionating component selected from the group consisting of salts, polymers, surfactants, or combinations thereof.

18. The method of claim 13, wherein the first, second and third polymer or surfactant components have molecular weights between 200 and 10000.

19. The method of claim 1, wherein the fluid mixture comprises at least one of blood, plasma, cells, exosomes, proteins, cell free DNA, RNA, and circulating tumor DNA.

20. The method of claim 1, wherein the nucleic acid target size is selected to be within a 100 bp range below 10,000 bp.

21. The method of claim 1, wherein the nucleic acid target size is selected to be within a 50 bp range below 1000 bp.

22. The method of claim 1, wherein said first and second phase forming polymer or surfactant components comprise at least one component selected from the group consisting of salts, polymers, surfactants, or combinations thereof.

23. A composition for isolating and concentrating nucleic acids of target sizes from a fluid mixture including nucleic acids and contaminants, comprising:
components for forming a first aqueous two-phase system (ATPS) from a first phase forming polymer or surfactant component dissolved in a first phase solution, and a second phase solution, such that when mixed with the fluid mixture target nucleic acid fragments below a target size partition to said second phase solution and contaminants partition to the first phase solution;
components for forming a second ATPS from a second phase forming polymer or surfactant component dissolved in a third phase solution and a fourth phase solution, such that when mixed with the second phase solution the target nucleic acid fragments partition to and concentrate in the third phase solution; and
materials for concentrating target nucleic acid fragments from the third phase solution.

24. A kit for isolating and concentrating nucleic acids of target sizes from a fluid mixture including nucleic acids and contaminants, comprising:
components for forming a first aqueous two-phase system (ATPS) from a first phase forming polymer or surfactant component dissolved in a first phase solution, and a second phase solution, such that when mixed with the fluid mixture target nucleic acid fragments below a target size partition to said second phase solution and contaminants partition to the first phase solution, supported in a container;
components for forming a second ATPS from a second phase forming polymer or surfactant component dissolved in a third phase solution and a fourth phase solution, such that when mixed with the second phase solution the target nucleic acid fragments partition to and concentrate in the third phase solution, supported in a container; and
materials for concentrating target nucleic acid fragments from the third phase solution, supported in a container.
